(19) United States
(12) Patent Application Publication
(10) Pub. No.: US 2003/0100762 A1
(43) Pub. Date: May 29, 2003

(54) Fluorescent dyes

(76) Inventors: Gregory Kaler, San Diego, CA (US); Clinton A. Threlfall, San Diego, CA (US); Channa Basava, San Diego, CA (US); Ilya Okun, San Diego, CA (US)

Correspondence Address: David B. Waller, David B. Waller & Associates, Suite 214, 5677 Oberlin Drive, San Diego, CA, 92121, US

(21) Appl. No.: 10/227,466

(22) Filed: Aug. 23, 2002

Related U.S. Application Data
(60) Provisional application No. 60/315,276, filed on Aug. 27, 2001.

(51) Int. Cl. C 07D 4 3/02

(52) U.S. Cl. 544/296

(57) ABSTRACT

Compounds of the formula (I) are provided, wherein n is 1 or 2, Q₁ and Q₂ are each individually selected from the group consisting of O and S, X is H or halogen, and R₁, R₂, R₃ and R₄ are each individually selected from the group consisting of hydrogen and uncharged groups having from 1 to about 20 carbon atoms. Methods of making such compounds are also described. Such compounds are useful as fluorescent dyes.

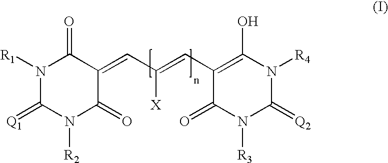

### RELATED APPLICATION INFORMATION

This application claims priority to U.S. Provisional Patent Application No. 60/315,276, filed Aug. 27, 2001, which is hereby incorporated by reference in its entirety.

### BACKGROUND OF THE INVENTION

1. Field of the Invention

This invention relates to dyes, and particularly to membrane potential sensitive fluorescent dyes useful in cell biology.

2. Description of the Related Art

Certain bis-barbituric acid oxonols have been used for monitoring plasma membrane potentials by changes in fluorescence intensity upon cell membrane depolarization or hyperpolarization, see Epps et al., Chem. Phys. Lipids 69:137-150 (1994); Brauner et al., Biochim. Biophys. Acta. 771:208-216 (1984). U.S. patent application Ser. No. 09/535,261, which is incorporated by reference herein in its entirety, describes methods of identifying compounds having biological activity using membrane penetrative membrane potential sensitive fluorescent dyes. However, the number of available dyes known to be useful for such applications is limited. Thus, there is a need for membrane penetrative dyes and particularly for fluorescent dyes sensitive to membrane potentials.

### SUMMARY OF THE INVENTION

A preferred embodiment provides compounds of the formula (I)

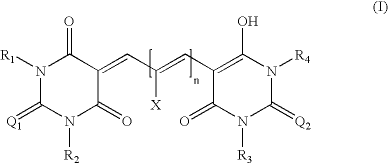

wherein n is 1 or 2, Q₁ and Q₂ are each individually selected from the group consisting of O atom and S atom, X is H or halogen, and R₁, R₂, R₃ and R₄ are each individually selected from the group consisting of hydrogen, allyl, alkyl having from 1 to 20 carbon atoms, alkoxycarbonyl having from 1 to 20 carbon atoms, and aryl having from 6 to 10 carbon atoms,

with the proviso that, if n is 2, Q₁ and Q₂ are O and X is H, then R₁, R₂, R₃ and R₄ are not all n-butyl; and with the proviso that, if n is 1, Q₁ and Q₂ are S and X is H, then R₁, R₂, R₃ and R₄ are not all ethyl and are not all n-butyl; and with the proviso that, if n is 1, Q₁ and Q₂ are O and X is H, then R₁, R₂, R₃ and R₄ are not all n-butyl.

Methods for making compounds of the formula (I) are also provided. These and other embodiments are described in greater detail below.

### BRIEF DESCRIPTION OF THE DRAWINGS

These and other aspects of the invention will be readily apparent from the following description and from the appended drawings, which are meant to illustrate and not to limit the invention, and wherein:

FIG. 1 is a reaction scheme illustrating a preferred method for making an unsymmetrical compound of the formula (I).

FIG. 2 is a reaction scheme illustrating a preferred method for making a symmetrical compound of the formula (I).

FIGS. 3A and 3B are plots of the fluorescence kinetics of various dyes in PC12 cells.

### DETAILED DESCRIPTION OF THE PREFERRED EMBODIMENTS

A preferred embodiment is directed to compounds represented by the chemical formula (I):

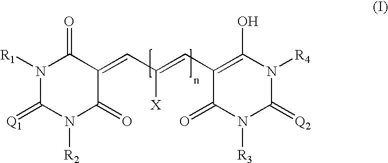

wherein n is 1 or 2, Q₁ and Q₂ are each individually selected from the group consisting of O and S, X is H or halogen, and R₁, R₂, R₃ and R₄ are each individually selected from the group consisting of hydrogen and uncharged groups containing from 1 to about 20 carbon atoms. Examples of such uncharged groups include allyl, alkyl having from 1 to about 20 carbon atoms, alkylidene having from 1 to about 20 carbon atoms, alkanol having from 1 to about 20 carbon atoms, alkylene ether having from 1 to about 20 carbon atoms, halogen-substituted alkyl having from 1 to about 20 carbon atoms, cycloalkyl having from about 5 to about 10 carbon atoms, mixed alkyl-cycloalkyl having from 1 to 20 carbon atoms, alkoxycarbonyl having from 1 to 20 carbon atoms, aryl having from 6 to 10 carbon atoms, and substituted aryl having from 6 to 20 carbon atoms, wherein the aryl group can have one or more substituents selected from the group consisting of hydroxyl, ester, alkyl, alkyl ether, and halogen.

Preferably, the compounds represented by the formula (I) are fluorescent dyes, more preferably fluorescent dyes that display sensitivity to electric potentials across cell membranes.

Preferred compounds of the formula (I) are those in which R₁, R₂, R₃ and R₄ are each individually selected from the group consisting of hydrogen, allyl, methyl, ethyl, propyl, butyl, phenyl, and —CH₂CO₂CH₂CH₃ (CH₂CO₂Et). Preferably, if n is 2, Q₁ and Q₂ are O and X is H, then R₁, R₂, R₃ and R₄ are not all n-butyl. Also, if n is 1, Q₁ and Q₂ are S and X is H, then R₁, R₂, R₃ and R₄ are preferably not all ethyl and are preferably not all n-butyl. Likewise, if n is 1, Q₁ and Q₂ are O and X is H, then R₁, R₂, R₃ and R₄ are preferably not all n-butyl. Compounds 1-32 in Table 1 are specific examples of chemical compounds having the formula (I).

TABLE 1

| No. | n | X | Q₁ | Q₂ | R₁ | R₂ | R₃ | R₄ |
|---|---|---|---|---|---|---|---|---|
| 1 | 1 | H | S | S | methyl | methyl | methyl | methyl |
| 2 | 1 | H | S | S | H | methyl | methyl | methyl |
| 3 | 1 | H | S | S | methyl | methyl | ethyl | ethyl |
| 4 | 1 | Cl | S | S | methyl | methyl | methyl | methyl |
| 5C | 1 | H | S | S | ethyl | ethyl | ethyl | ethyl |
| 6 | 1 | H | S | S | methyl | methyl | allyl | H |
| 7 | 1 | H | O | O | n-butyl | CH₂CO₂Et | n-butyl | CH₂CO₂Et |
| 8C | 1 | H | O | O | n-butyl | n-butyl | n-butyl | n-butyl |
| 9 | 1 | H | S | S | ethyl | ethyl | H | allyl |
| 10 | 1 | H | O | O | n-propyl | n-propyl | n-propyl | n-propyl |
| 11 | 1 | H | O | O | phenyl | phenyl | phenyl | phenyl |
| 12 | 1 | H | S | S | n-butyl | n-butyl | H | allyl |
| 13 | 1 | H | S | S | phenyl | phenyl | phenyl | phenyl |
| 14 | 2 | H | S | S | ethyl | n-butyl | methyl | methyl |
| 15 | 2 | H | S | S | phenyl | phenyl | phenyl | phenyl |
| 16 | 2 | H | S | S | n-propyl | n-propyl | n-propyl | n-propyl |
| 17 | 2 | H | O | O | n-propyl | n-propyl | n-propyl | n-propyl |
| 18 | 2 | H | O | O | CH₂CO₂Et | n-butyl | H | methyl |
| 19 | 2 | H | S | S | allyl | H | allyl | H |
| 20 | 2 | H | S | S | ethyl | ethyl | ethyl | ethyl |
| 21 | 2 | H | O | O | phenyl | phenyl | phenyl | phenyl |
| 22 | 2 | H | S | S | H | ethyl | H | methyl |
| 23 | 2 | H | S | S | ethyl | H | ethyl | H |
| 24 | 2 | H | S | S | methyl | methyl | methyl | methyl |
| 25C | 2 | H | O | O | n-butyl | n-butyl | n-butyl | n-butyl |
| 26 | 2 | H | S | S | phenyl | phenyl | methyl | H |
| 27 | 2 | H | S | S | methyl | H | methyl | H |
| 28 | 2 | H | O | O | methyl | methyl | methyl | methyl |
| 29 | 2 | H | O | O | n-butyl | CH₂CO₂Et | n-butyl | CH₂CO₂Et |
| 30 | 2 | H | S | S | methyl | methyl | methyl | H |
| 31 | 2 | H | S | S | n-butyl | n-butyl | n-butyl | n-butyl |
| 32 | 2 | H | O | O | ethyl | ethyl | ethyl | ethyl |

Another preferred embodiment is directed to compounds represented by the chemical formula (II):

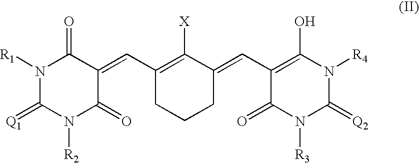

wherein Q₁ and Q₂ are each individually selected from the group consisting of O and S, X is H or halogen, and R₁, R₂, R₃ and R₄ are each individually selected from the group consisting of hydrogen and uncharged groups containing from 1 to about 20 carbon atoms. Examples of such uncharged groups include allyl, alkyl having from 1 to about 20 carbon atoms, alkylidene having from 1 to about 20 carbon atoms, alkanol having from 1 to about 20 carbon atoms, alkylene ether having from 1 to about 20 carbon atoms, halogen-substituted alkyl having from 1 to about 20 carbon atoms, cycloalkyl having from about 5 to about 10 carbon atoms, mixed alkyl-cycloalkyl having from 1 to 20 carbon atoms, alkoxycarbonyl having from 1 to 20 carbon atoms, aryl having from 6 to 10 carbon atoms, and substituted aryl having from 6 to 20 carbon atoms, wherein the aryl group can have one or more substituents selected from the group consisting of hydroxyl, ester, alkyl, alkyl ether, and halogen.

Preferably, the compounds represented by the formula (II) are fluorescent dyes, more preferably fluorescent dyes that display sensitivity to electric potentials across cell membranes.

Preferred compounds of the formula (II) are those in which R₁, R₂, R₃ and R₄ are each individually selected from the group consisting of hydrogen, allyl, methyl, ethyl, propyl, butyl, phenyl, and CH₂CO₂Et. Compound No. 33 (continuing the numbering scheme shown in Table 1) is a specific example of a preferred compound of the formula (II) in which Q₁ and Q₂ are each S, X is Cl, R₁ and R₃ are each allyl, and R₂ and R₄ are each H.

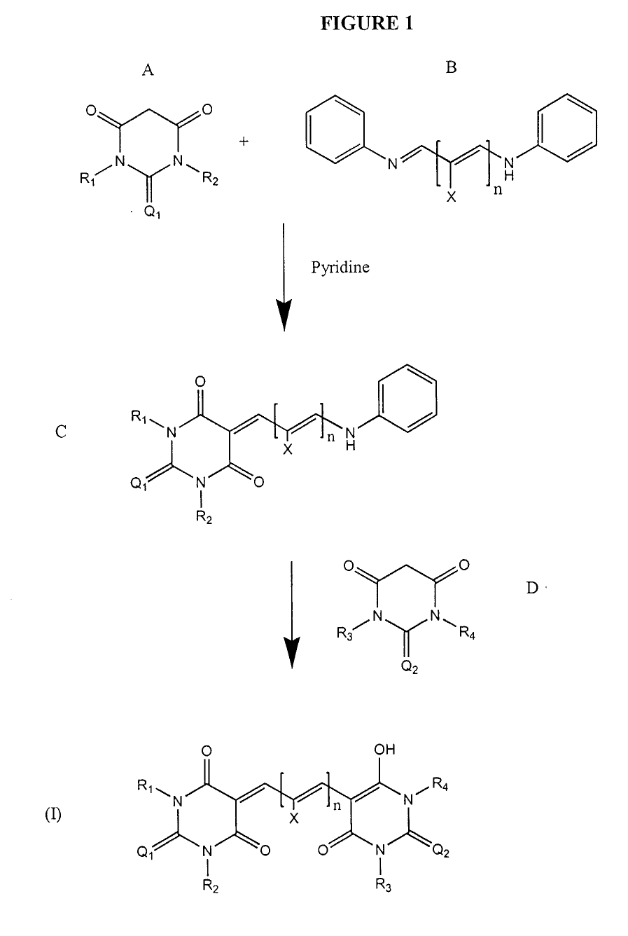

A general reaction scheme for making the compounds of the formula (I) is illustrated in FIG. 1. A barbituric acid or thiobarbituric acid compound of the general formula A, where R₁, R₂ and Q₁ are the same as described previously, is reacted with a compound of the formula B where n is 1 or 2 and X is H or halogen. Such compounds may be obtained from commercial sources or synthesized by methods known to those skilled in the art, see, e.g., A. I. Vogel, “Practical Organic Chemistry,” Longman Group Limited, London, (1978) p. 905; F. A. Carey, R. J. Sundberg, “Advanced Organic Chemistry, Part A” Plenum Press, New York (1990), p. 477. Preferably, compound B is N-(5-(phenylamino)-2,4-penta-dienylidene)aniline or N-(5-(phenylamino)-2-propenylidene)aniline.

Preferred reaction conditions for conducting the reaction illustrated in FIG. 1 are as follows: Compounds A and B are mixed together for a period of time and at a temperature effective to produce the product C, preferably a temperature in the range of about 20° C. to about 100° C. for a period of time in the range of from about 10 minutes to about 5 hours, preferably in the presence of an organic base, more preferably in the presence of pyridine. An excess amount of compound B is preferably used as compared to the amount of compound A, more preferably in the range of about 5× to about 15× excess, in order to increase yields of the mono-substituted product C. Since the reaction product usually contains some amount of di-substituted product, the reaction product is preferably at least partially purified by extraction and/or column chromatography to increase the purity of product C. Product C is then stirred and preferably heated with an approximately equimolar amount of reagent D for a period of time and at a temperature effective to produce a product containing the compound of the formula (I), preferably a temperature in the range of about 20° C. to about 100° C. for a period of time in the range of from about 10 minutes to about 5 hours. The resulting compound of the formula (I) is preferably purified by washing the reaction product with methylene chloride and then subjecting it to column chromatography, preferably using methylene chloride/methanol as eluant.

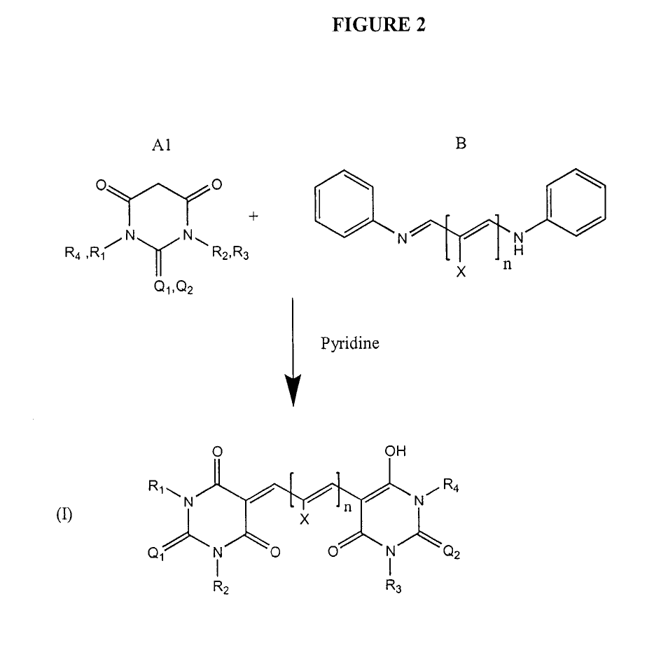

FIG. 2 illustrates a single step reaction sequence that is preferred when preparing symmetrical compounds of the formula (I) in which R₁=R_{4,} R₂=R₃ and Q₁=Q₂. Compounds A1 and B are preferably mixed together for a period of time and at a temperature effective to produce a symmetrical compound of the formula (I), preferably a temperature in the range of about 20° C. to about 100° C. for a period of time in the range of from about 10 minutes to about 5 hours, preferably in the presence of an organic base, more preferably in the presence of pyridine. The compound of the formula (I) is preferably purified by washing the resulting reaction product with methylene chloride and then subjecting it to column chromatography, preferably using methylene chloride/methanol as eluant.

The compounds of the formula (II) may be prepared in an analogous manner, except that corresponding compounds of the formula B1 are preferably used in place of compounds of the formula B:

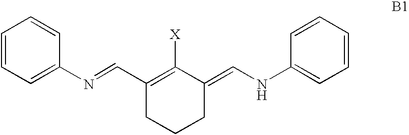

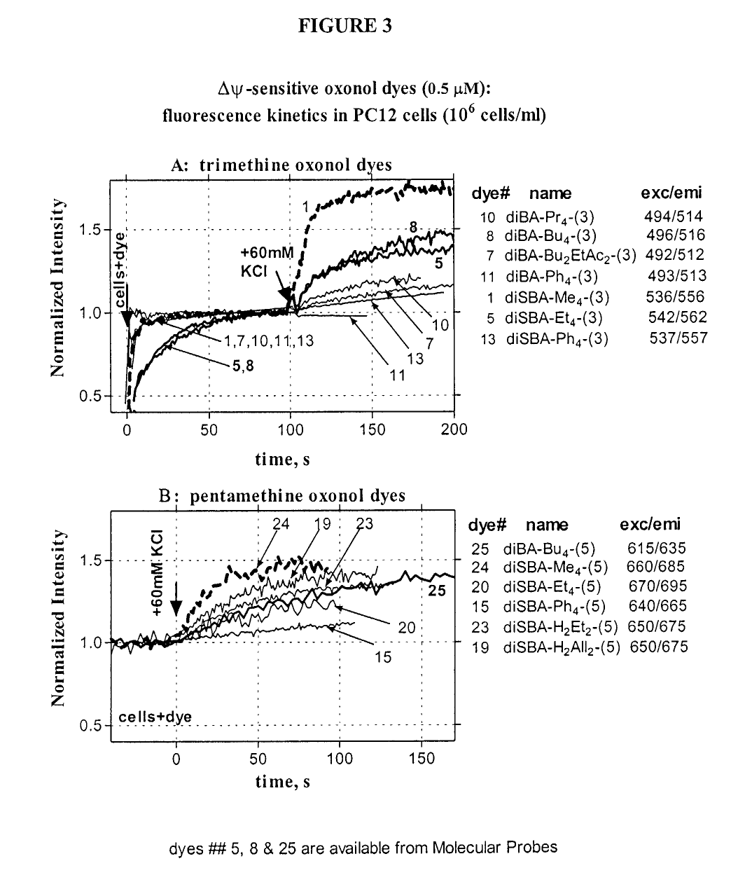

The compounds of the formulas (I) and (II) are fluorescent dyes. These dyes are useful in a broad range of applications and are particularly useful for detecting electric potentials across cell membranes. FIGS. 3A and 3B illustrate the kinetics of the fluorescent response of the indicted fluorescence dyes to KCl-induced depolarization of PC12 cells. These results were obtained as described in the Examples below.

### EXAMPLE 1

The 1,3-diethythiobarbituric acid used in Example 2 was prepared as follows: Into a 250 milliliter (mL) round-bottomed flask equipped with a reflux condenser was added 150 mL of a 21% solution of sodium ethoxide in ethanol (40 millimoles (mmol)). About 3.2 grams (20 mmol) of diethylmalonate was then added, followed by 20 mL of a hot ethanol (about 70° C.) solution containing about 2.08 grams (20 mmol) of 1-allyl-2-thiourea. The resulting mixture was stirred and refluxed for about 7 hours. About 200 mL of hot water was added, followed acidification with dilute HCl. The resulting mixture was filtered hot to remove the solids, and the filtrate was allowed to cool overnight in a refrigerator at about 4° C. The resulting precipitate was collected by filtration, washed with ice water, and dried.

### EXAMPLE 2

Compound No. 20 was prepared as follows: About 200 milligrams (about 1 mmol) of 1,3-diethythiobarbituric acid (prepared as in Example 1) and about 142 milligrams (about 0.5 mmol) of N-[5-(phenylamino)-2,4-penta-dienylidene]aniline monohydrochloride were added to a round-bottomed flask equipped with a stir bar. About one mL of pyridine was added, a reflux condenser was attached, and the mixture was stirred for about 1.5 hours. The pyridine was then removed by rotary evaporation under reduced pressure. The resulting residue was suspended in a few milliliters of methylene chloride and filtered. The filtrate was subjected to column chromatography (silica gel column with a 97:3 methylene chloride/methanol solvent mixture). The structure of Compound No. 20 was confirmed by fluorescence spectroscopy.

### EXAMPLE 3

These results shown in FIGS. 3A and 3B were obtained using PC12 cells (3 ml suspension in the hybridoma medium, 10⁶ cells/ml) that were contained in a quartz cuvette inserted into the a cuvette holder in a FluoroMax2 spectrofluorometer, with constant magnet stirring. PC12 cells are rat adrenal pheochromocytoma cells and were obtained commercially from American Type Culture Collection.

FIG. 3A shows the results obtained using the trimethine dyes (n=1 in formula (I)). The indicated trimethine dyes were added at zero time into the cuvette as 0.5 mM solutions in DMSO (3 μL) to a final concentration of 0.5 μM. Dyes were allowed to equilibrate with cells for 100 seconds, and then 60 mM KCl was added (60 μL of 3 M solution in water). The results show that compound No. 1 equilibrated faster with cells and displayed significantly higher sensitivity to membrane potential than the commercially available trimethine dyes (No. 5 and No. 8).

FIG. 3B shows the results obtained using the pentamethine dyes (n=2 in formula (I)). The indicated pentamethine dyes equilibrate with cells more slowly than trimethine dyes. Therefore, pentamethine dyes were preincubated with cells for about 10 minutes before measurement. The results show that compound No. 24 displayed significantly higher sensitivity to membrane potential as compared to the commercially available pentamethine dye No. 25.

It will be appreciated by those skilled in the art that various omissions, additions and modifications may be made to the processes described above without departing from the scope of the invention, and all such modifications and changes are intended to fall within the scope of the invention, as defined by the appended claims.

What is claimed is:

1. A compound of the formula (I) 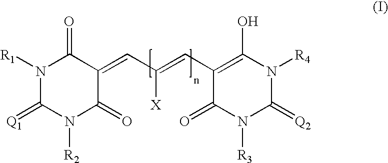
wherein
n is 1 or 2, Q₁ and Q₂ are each individually selected from the group consisting of O and S, X is H or halogen, and R₁, R₂, R₃ and R₄ are each individually selected from the group consisting of hydrogen, allyl, alkyl having from 1 to 20 carbon atoms, alkoxycarbonyl having from 1 to 20 carbon atoms, and aryl having from 6 to 10 carbon atoms,
with the proviso that, if n is 2, Q₁ and Q₂ are O and X is H, then R₁, R₂, R₃ and R₄ are not all n-butyl; and with the proviso that, if n is 1, Q₁ and Q₂ are S and X is H, then R₁, R₂, R₃ and R₄ are not all ethyl and are not all n-butyl; and with the proviso that, if n is 1, Q₁ and Q₂ are O and X is H, then R₁, R₂, R₃ and R₄ are not all n-butyl.

2. The compound of claim 1 wherein R₁, R₂, R₃ and R₄ are each individually selected from the group consisting of hydrogen, allyl, methyl, ethyl, propyl, butyl, phenyl, and CH₂CO₂Et.

3. The compound of claim 1 wherein n is 1, X is H, Q₁ is S, Q₂ is S, R₁ is methyl, R₂ is methyl, R₃ is methyl and R₄ is methyl.

4. The compound of claim 1 wherein n is 1, X is H, Q₁ is O, Q₂ is O, R₁ is n-butyl, R₂ is CH₂CO₂Et, R₃ is n-butyl and R₄ is CH₂CO₂Et.

5. The compound of claim 1 wherein n is 1, X is H, Q₁ is O, Q₂ is O, R₁ is n-propyl, R₂ is n-propyl, R₃ is n-propyl and R₄ is n-propyl.

6. The compound of claim 1 wherein n is 1, X is H, Q₁ is O, Q₂ is O, R₁ is phenyl, R₂ is phenyl, R₃ is phenyl and R₄ is phenyl.

7. The compound of claim 1 wherein n is 2, X is H, Q₁ is S, Q₂ is S, R₁ is phenyl, R₂ is phenyl, R₃ is phenyl and R₄ is phenyl.

8. The compound of claim 1 wherein n is 2, X is H, Q₁ is S, Q₂ is S, R₁ is allyl, R₂ is H, R₃ is allyl and R₄ is H.

9. The compound of claim 1 wherein n is 2, X is H, Q₁ is S, Q₂ is S, R₁ is ethyl, R₂ is ethyl, R₃ is ethyl and R₄ is ethyl.

10. The compound of claim 1 wherein n is 2, X is H, Q₁ is S, Q₂ is S, R₁ is ethyl, R₂ is H, R₃ is ethyl and R₄ is H.

11. The compound of claim 1 wherein n is 2, X is H, Q₁ is S, Q₂ is S, R₁ is methyl, R₂ is methyl, R₃ is methyl and R₄ is methyl.

12. A method of making the compound of claim 1, comprising
mixing together a compound of the formula A with an excess amount, on a molar basis, of a compound of the formula B, for a period of time and at a temperature effective to form a compound of the formula C; and 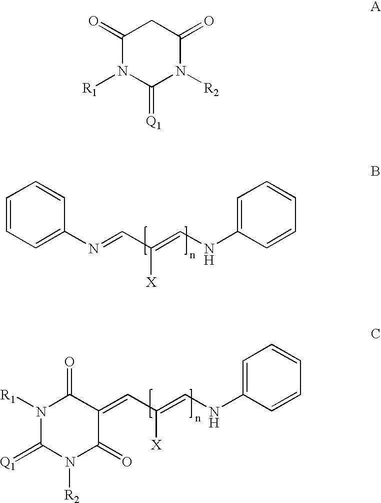
mixing together the compound of the formula C with a compound of the formula D, 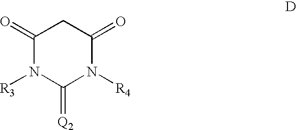
for a period of time and at a temperature effective to form the compound of the formula (I), wherein n is 1 or 2, Q₁ and Q₂ are each individually selected from the group consisting of O and S, X is H or halogen, and R₁, R₂, R₃ and R₄ are each individually selected from the group consisting of hydrogen, allyl, alkyl having from 1 to 20 carbon atoms, alkoxycarbonyl having from 1 to 20 carbon atoms, and aryl having from 6 to 10 carbon atoms.

13. A method of making the compound of claim 1, comprising
mixing together a compound of the formula A1 with a compound of the formula B, for a period of time and at a temperature effective to form a compound of the formula (I), 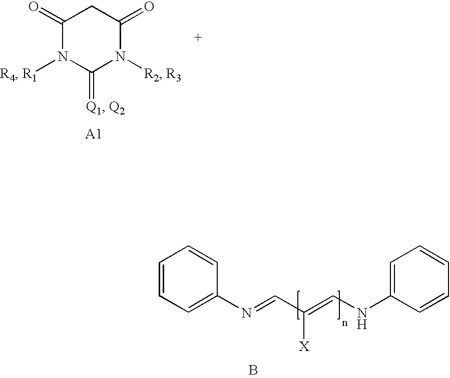
wherein n is 1 or 2, Q₁ and Q₂ are both either O or S, X is H or halogen, and R₁=R₄ are selected from the group consisting of hydrogen, allyl, alkyl having from 1 to 20 carbon atoms, alkoxycarbonyl having from 1 to 20 carbon atoms, and aryl having from 6 to 10 carbon atoms, and R₂=R₃ are selected from the group consisting of hydrogen, allyl, alkyl having from 1 to 20 carbon atoms, alkoxycarbonyl having from 1 to 20 carbon atoms, and aryl having from 6 to 10 carbon atoms.
